(19) United States
(12) Patent Application Publication
(10) Pub. No.: US 2012/0213715 A1
(43) Pub. Date: Aug. 23, 2012

(54) Method and composition for the prevention and treatment of early childhood dental caries

(75) Inventors: Chifu Huang, Lexington, KY (US); Jeffrey Lee Ebersole, Lexington, KY (US)

(73) Assignee: Chifu Huang, Lexington, KY (US)

(21) Appl. No.: 13/033,594

(22) Filed: Feb. 23, 2011

(51) Int. Cl. A61K 8/92; A61Q 11/00

(52) U.S. Cl. 424/49

(57) ABSTRACT

The present invention discloses a composition of omega polyunsaturated fatty acids for controlling and preventing early childhood dental caries. The application of this composition can be used for the preparation of drugs, nutritional supplements, food and daily necessities with oral health care and therapeutic effects for children.

### FIELD OF THE INVENTION

The present invention relates to a composition of fatty acids, which include omega-3, omega-6, and omega-9 fatty acids, for their anti-microbial activity. Specifically, the omega-3 (DHA), omega-6 fatty acids (ARA, GLA), and omega-9 fatty acid (oleic acid, OA), having antimicrobial activity against cariogenic pathogens, and their application in the prevention and treatment of early childhood dental caries.

### BACKGROUND OF THE INVENTION

Omega-3 fatty acids include docosahexaenoic acid (DHA) and α-linolenic acid (ALA) and others, belonging to n-3 unsaturated fatty acids (also known as the Ω-3 unsaturated fatty acids or omega-3 fatty acid) family. Their common feature, the first double bond in the carbon chain located at third position, has an important role in physiological functions. Other omega-6 fatty acids, such as arachidonic acid (ARA) and γ-linolenic acid (GLA), and omega-9 fatty acid (oleic acid), also have important physiological functions.

For example, docosahexaenoic acid (DHA) is required for retinal development and normal functions. The study indicated that during the critical period of development, a deficiency of DHA can result in permanent defects in retinal function. In addition, DHA plays a crucial role of the nervous system function because it can protect nerve cells (neurons) to avoid programmed cell death (from apoptosis), and enhance the vitality of the nervous system. In general, DHA is important because it not only inhibits the promotion of prostaglandin and thromboxane formation (in order to protect vascular endothelial cells, inhibit platelet aggregation, prevent atherosclerotic plaque and vascular thrombosis), but is also essential to the fetus and the normal development of infants and young children.

A recent world-wide survey reported that 80% of the world population in both developed and undeveloped countries suffer some level of dental caries. Dental caries is the predominant cause of tooth loss in children and young adults, as well as an increasing presence in the elderly (ie. root caries) and remains the most prevalent of all chronic diseases among children in the US, 5× more common than asthma and 7× more common than hay fever. Nearly 20% of all children by 2-4 years of age, >50% by 6-8 years, and over ¾ of adolescents by age 17 are affected. There is no effective method to prevent and treat the child caries.

Due to essential fatty acids found in fish oil, these fatty acids are widely used in functional foods. However, no combination of the fatty acids has been proposed or used to prevent or treat early childhood dental caries. The current invention utilizes the novel discovery of anti-microbial activity of fatty acids, which can be applied for controlling and preventing dental caries. There is a great need for this, since as many as 50% of children suffer from untreated dental caries in the U.S. There is no prior use of the fatty acid combination in the prevention of dental caries.

Provided by the invention of omega-3, omega-6, and omega-9 fatty acids, having anti-microbial activity for the prevention and treatment of caries disease in children, is the first time that the composition of fatty acids used against cariogenic pathogens, such as Streptococcus mutans and Lactobacillius, due to their synergistic effect of the fatty acids. The application prospects have important social, economic and health benefits, while the compositions can meet the market need.

### DISCLOSURE OF INVENTION

The purpose of the present invention is to provide compositions and novel application of omega-3, omega-6, and omega-9 fatty acids, their methyl esters, and ethyl esters, to ensure the full exploitation of the anti-cariogenic activity for improving children's health. The characteristics are the discovery of antimicrobial activity of omega-3, omega-6, and omega-9 fatty acids against cariogenic bacteria and their application for dental caries. Specifically, the fatty acids, fatty acid methyl esters, and fatty acid ethyl esters found in fish oil or algae or plants, have significant anti-microbial activity, allowing them to be applied to the prevention and treatment of childhood caries.

The composition of the fatty acids can also be used in combination with vitamins A, C, E and D; and sweetened with Lo Han Kuo extract. The Lo Han Kuo extract has anti-cariogenic activity. It can also be used with other sweeteners, such as stevia and xylitol.

The invention will make the methods for the application of antimicrobial activity of common omega-3 fatty acid (DHA) and omega-6 fatty acid (ARA, GLA), and omega-9 fatty acid (OA), their fatty acid methyl esters, and fatty acid ethyl esters and their application of prevention of early childhood caries disease. The characteristics of the above methods and compounds are:
(1) Fatty acids, docosahexaenoic acid (DHA), arachidonic acid (ARA) and γ-linolenic acid (GLA), and oleic acid (OA), and also their fatty acid methyl esters, and fatty acid ethyl esters have novel antimicrobial activity against cariogenic pathogens. The compositions of the above fatty acids have stronger antimicrobial bioactivity against cariogenic pathogens, such as, Streptococcus mutans and are suitable for children consumption.
(2) The composition of the omega-3 and omeg-6 fatty acids, fish oil fatty acid methyl ester, and fish oil fatty acid ethyl ester are to be used in prevention and treatment of childhood caries diseases. The present invention provides an application for childhood dental caries prevention and control by omega-3 and omega-6 fatty acids, their fatty acid methyl esters and ethyl esters. The method for prevention and control of the oral infection mentioned above is that omega-3, omega-6, and omega-9 fatty acids, their fatty acid methyl esters, and fatty acid ethyl esters are prepared for oral health care, 0.01-20% of the content to the final product weight, where the specific content items based on the type of system and requirements. These concentrations can provide children with the necessary nutrition for development as well as with the dental health benefits. But they are used not to exceed the daily input requirement (220 mg).

The inventor verified the test by dissolving 1 mg of omega-3 fatty acid (DHA) and omega-6 fatty acids (ARA, GLA), omega-9 fatty acid (oleic acid) in 1000 μl ethanol solution, and mix them up. Adding took 1 μl (final concentration 1 μg/ml) and 5 μl (final concentration 5 μg/ml) of the mixture solution to the well of 96-well plate containing 100 μA of Streptococcus mutans in medium and cultured overnight at 37° C. After the overnight culture, the medium was diluted 1:10000 times, 5 μl of the diluted culture was applied to blood agar plate and cultured overnight. Calculated the number of colonies on the plates and compared with the control group to obtain the percentage of inhibition. It was found that the 1 μg/ml final concentration of the composition could inhibit at least 50% of Streptococcus mutans growth.

The composition of present invention, using the novel anti-microbial activity of omega-3, omega-6, and omega-9 fatty acids, show strong activity against cariogenic pathogens, and can be applied in products for oral health care and as a preventive solution for early childhood dental caries.

The composition of the fatty acids can be used in combination with vitamins A, C, E and D; and sweetened with Lo Han Kuo extract. The Lo Han Kuo extract has anti-cariogenic activity. The composition can also be used with other sweeteners, such as stevia and xylitol.

The composition can be used in medicinal formulations such as tablets, sprays, powder, film, paint, ointment, gel, liquid, foam, mouthwash, etc. The treatments mainly refers to the tablets for oral medication in various form of tablets, including oral tablets, bio-adhesive patches, dispersible tablets, effervescent tablets, chewable tablets, tablets and other solutions. The fish oil fatty acids, fish oil fatty acid methyl ester, and fish oil fatty acid ethyl ester in the above-mentioned composition should account for at least 0.1 percent to the weight of the mixture of drugs. The more effective composition would generally be 0.01-20%.

The composition can be used in dietary supplements, as well as ingredients in foods such as baby formula, candy, chewing gum, beverages, and other dairy products. The fish oil fatty acids, fish oil fatty acid methyl ester, and fish oil fatty acid ethyl ester content should be at least 0.01% to weight mixture of food, although 0.01-20% would be ideal.

The composition can be used in oral daily necessities such as baby toothpaste, mouthwash. The fish oil fatty acids, fish oil fatty acid methyl ester, and fish oil fatty acid ethyl ester content should be at least 0.01% to the weight of a mixture of daily necessities, although 0.01-20% would be ideal.

### EXAMPLES

The following examples are presented by way of illustration, not of limitation.

### Example 1

Dissolve 1 mg fatty acids, DHA, ARA, GLA, and OA in 10 ml ethanol to make a 1 mg/ml stock solution; and mix them up. Take 1 μl and 5 μl of mixture solution and add into 100 μl Streptococcus mutans culture (at final concentration 1 μg/ml and 5 μg/ml), incubate the said bacterial culture overnight at 37° C. Afterwards, dilute the overnight culture by 1:10000. Five μl of the diluted culture mix was applied to blood agar plate and cultured overnight. Calculate the colony numbers on the plate and compared with the negative control plate. Final concentration of 1 μg/ml of the mixture composition could significantly inhibit the growth of Streptococcus mutans. The result shows that the IC₅₀ of the fatty acid composition is approximately 1 μg/ml.

### Example 2

Dissolve 1 mg of omega-3 fatty acid ethyl ester DHA-EE, ARA-EE, GLA-EE in 10 ml ethanol, and mix them up. One μl and 5 μl of the above mixture solution were added into 100 μl of Streptococcus mutans culture (at final concentration 1 μg/ml and 5 μg/ml), and cultured overnight at 37° C. Dilute this culture by 1:10000. Five μl of diluted solution was applied to blood agar plate and cultured overnight. Calculate the clone number on the disk, and compared with the negative control. Results showed that fish oil ethyl esters have significant anti-bacterial activity. Final concentration of 1 μg/ml extract could inhibit the growth of Streptococcus mutans. The result shows that the mixture fatty acid ethyl ester IC₅₀ is approximately 2.5 μg/ml.

### Example 3

Dissolve 1 mg of omega-3 fatty acid methyl ester DHA-ME, ARA-ME, GLA-ME, OA-ME in 10 ml ethanol, and mix them up. One μl and 5 μl of the mixture solution were added into 100 μl Streptococcus mutans solution (at final concentration 1 μg/ml and 5 μg/ml), and then cultured overnight at 37° C. Dilute this culture by 1:10000 and apply 5 μl of diluted solution to blood agar plate, and cultured overnight. Calculate the clone number on the plate and compare with the negative control. Results showed that the omega-3 fatty acid methyl esters have significant anti-bacterial activity. Final concentration of 1 μg/ml omega-3 fatty acid methyl ester could inhibit the growth of Streptococcus mutans. The study showed that the fatty acid methyl ester composition have IC₅₀ of approximately 2.5 μg/ml.

### Example 4

The weight percentage composition of fatty acids DHA, ARA, GLA, OA or their fatty acid methyl ester/or fatty acid ethyl ester, is used in baby formula as follows:

| | |
|---|---|
| The fatty acid composition | 1-5% |
| Milk | 90-95% |
| Mannitol, or xylitol or sorbitol or syrup | 1-5% |
| Or high intense sweetener Lo Han Kuo extract | 0.5~1% |

### Example 5

The weight percentage composition of dairy products, containing fatty acid DHA, ARA, GLA, OA, or their fatty acid methyl ester/or fatty acid ethyl ester, is as follows:

| | |
|---|---|
| The fatty acid composition | 1-5% |
| Milk | 90-95% |
| Mannitol, or xylitol or sorbitol or syrup | 1-5% |
| Or high intense sweetener | 0.5~1% |

### Example 6

The weight percentage composition of chewing gum, containing fish oil fatty acids (DHA, GLA, ARA, OA)/or their fatty acid methyl ester/or fatty acid ethyl ester, is as follows:

| | |
|---|---|
| The fatty acid composition | 1-5% |
| Gum base | 80% |
| Mannitol, or xylitol or sorbitol | 10% |
| Menthol | 0.2% |
| Lecithin | 0.6% |
| Edible flavors (such as banana or other flavors) | 0.9% |
| Edible citric acid | 0.2% |

### Example 7

The weight percentage composition of chewing gum, containing fatty acids (DHA, ARA, GLA, OA)/or fatty acid methyl esters/or fatty acid ethyl esters, is as follows:

| | |
|---|---|
| The fatty acid composition | 1-5% |
| Gum base | 80% |
| High intense sweetener mogroside, rebaudisoside | 2% |
| Menthol | 0.2% |
| Lecithin | 0.6% |
| Edible flavors (such as banana or other flavors) | 0.9% |
| Edible citric acid | 0.2% |

### Example 8

The weight percentage composition of candy, containing fatty acids (DHA, ARA, GLA, OA),/or omega fatty acid methyl ester/ethyl esters, is as follows:

| | |
|---|---|
| The fatty acid composition | 1-5% |
| Milk powder | 5% |
| Lo Han Kuo extract | 1% |
| Dry starch syrup solid objects | 30~35% |
| Non-fat milk solids | 5~10% |
| Plant fat | 15-20% |
| Salt | 0.2-0.3% |
| Colloidal | 1.5-2.0% |

Even though specific examples of the invention are explicitly disclosed herein, the above specifications and specific examples are not restrictive and are illustrative. Upon the review of the specifications with the examples given herein, many variations of the invention will become apparent to those skilled in the art. Therefore, full scope of the invention should be determined by the specifications and by references to the examples along with their full scope equivalents, along with such variations. It is expected that those skilled in the art to employ such variations and the invention to be practiced otherwise than specifically described here. Therefore, the invention includes all modification and variations and equivalents of the subject matter as cited in the attached claims. Henceforth, any combination of the herein described elements in all possible variations is encompassed by the invention.

### BEST MODE FOR CARRYING OUT THE INVENTION

The preferred embodiments of this invention are described herein, which includes the best mode that is known to the inventors for implementing the invention. Even though the invention has been described with reference to particular embodiments, it is to be understood that these embodiments are merely illustrative of the applications and principles of the composition. Therefore, it is to be understood that many modifications may be made to the illustrative embodiments without departing from the spirit and the scope of the invention as defined in the attached claims.

### INDUSTRIAL APPLICABILITY

The antimicrobial compositions of this invention containing omega polyunsaturated fatty acids are capable of maintaining the original antimicrobial activity, and can be used in a variety of applications where antimicrobial activity is desired, such as in materials for consumer goods including baby formula, baby foods and beverages, functional foods, drugs. Methods of preparing such compositions, and using such compositions, for example, in the treatment and/or prevention of early childhood dental caries.

1-7. (canceled)

8. A composition for affecting antimicrobial activity against cariogenic pathogens, comprising:
at least one fatty acid/ester selected from: an omega-3 fatty acid/ester, an omega-6 fatty acid/ester, and an omega-9 fatty acid/ester to a subject,
wherein the fatty acid/ester is formulated in an oral health, medicinal, or nutritional food product.

9. A composition of claim 8, wherein the at least one fatty acid/ester is selected from: docosahexaenoic acid (DHA), γ-linolenic acid (GLA), arachidonic acid (ARA), oleic acid (OA), docosahexaenoic acid (DHA) methyl/or ethyl esters, γ-linolenic acid (GLA) methyl/or ethyl esters, arachidonic acid (ARA) methyl/or ethyl esters, and oleic acid (OA) methyl/or ethyl esters.

10. The composition of claim 9, wherein the at least one fatty acid/ester is docosahexaenoic acid (DHA), or docosahexaenoic acid (DHA) methyl ester, or docosahexaenoic acid (DHA) ethyl ester.

11. The composition of claim 10, wherein the oral health, medicinal, or nutritional food product is selected from a chewable/chewing gum, a chewable/chewing tablets, a toothpaste, a mouthwash, a spray, a powder, a film, a paint, an ointment, a gel, a liquid, a foam, a paste, a tablet, a gum, a solution, a baby formula, a baby toothpaste, a beverage, and a dairy product.

12. The composition of claim 10, and further comprising a second fatty acid/ester selected from: γ-linolenic acid (GLA), arachidonic acid (ARA), oleic acid (OA), γ-linolenic acid (GLA) methyl/or ethyl esters, arachidonic acid (ARA) methyl/or ethyl esters, and oleic acid (OA) methyl/or ethyl esters

13. The composition of claim 10, and further comprising at least one additional component, selected from: vitamin A, vitamin C, vitamin D, vitamin E, Lo Han Kuo extract, stevia, and xylitol.

14. The composition of claim 8, wherein the oral health, medicinal, or nutritional food product, is selected from a chewable/chewing gum, a chewable/chewing tablets, a toothpaste, a mouthwash, a spray, a powder, a film, a paint, an ointment, a gel, a liquid, a foam, a paste, a tablet, a gum, a solution, a baby formula, a baby toothpaste, a beverage, and a dairy product.

15. The composition of claim 8, wherein the fatty acids comprise about 0.01% to about 20% weight/weight of the composition.

16. A method of affecting antimicrobial activity against cariogenic pathogens in a subject, comprising:
administering a composition of claim 8 to a subject, wherein the subject has or is at risk of developing a carie caused by a cariogenic pathogen.

17. The method of claim 16, wherein the composition comprises at least one fatty acid/esters selected from: docosahexaenoic acid (DHA), γ-linolenic acid (GLA), arachidonic acid (ARA), oleic acid (OA), docosahexaenoic acid (DHA) methyl/or ethyl esters, γ-linolenic acid (GLA) methyl/or ethyl esters, arachidonic acid (ARA) methyl/or ethyl esters, and oleic acid (OA) methyl/or ethyl esters.

18. The method of claim 17, wherein the composition comprises docosahexaenoic acid (DHA), or docosahexaenoic acid (DHA) methyl ester, or docosahexaenoic acid (DHA) ethyl ester.

19. The method of claim 16, wherein the subject is identified has having or being at risk of developing a carie caused by Streptococcus mutans and Lactobacillus.

20. The method of claim 16, wherein the fatty acids/esters comprise about 0.01% to about 20% weight/weight of the composition.

21. The composition of claim 16, wherein the oral health, medicinal, or nutritional food product, is selected from a chewable/chewing gum, a chewable/chewing tablets, a toothpaste, a mouthwash, a spray, a powder, a film, a paint, an ointment, a gel, a liquid, a foam, a paste, a tablet, a gum, a solution, a baby formula, a baby toothpaste, a beverage, and a dairy product.

22. A method of affecting antimicrobial activity against cariogenic pathogens in a subject, comprising:
administering a composition comprising at least one fatty acid/ester selected from: an omega-3 fatty acid/ester, an omega-6 fatty acid/ester, and an omega-9 fatty acid/ester to a subject, wherein the subject has or is at risk of developing a carie caused by a cariogenic pathogen.

23. The method of claim 22, wherein the composition comprises at least one fatty acid/ester selected from: docosahexaenoic acid (DHA), γ-linolenic acid (GLA), arachidonic acid (ARA), Oleic acid (OA), docosahexaenoic acid methyl/ethyl esters, γ-linolenic acid methyl/ethyl esters, arachidonic acid methyl/ethyl esters, and Oleic acid methyl/ethyl esters.

24. The method of claim 23, wherein the composition comprises DHA or DHA methyl/ethyl esters.

25. The method of claim 24, wherein the composition further comprises a second fatty acid/ester selected from: γ-linolenic acid (GLA), arachidonic acid (ARA), oleic acid (OA), γ-linolenic acid (GLA) methyl/ethyl esters, arachidonic acid (ARA) methyl/ethyl esters, and oleic acid (OA) methyl/ethyl esters; and/or at least one additional component, selected from: vitamin A, vitamin C, vitamin D, vitamin E, Lo Han Kuo extract, stevia, and Xylitol.

26. The method of claim 24, wherein the subject is identified has having or being at risk of developing a carie caused by Streptococcus mutans and Lactobacillus.

27. The method of claim 22, wherein the composition is formulated in a chewable/chewing gum, a chewable/chewing tablets, a toothpaste, a mouthwash, a spray, a powder, a film, a paint, an ointment, a gel, a liquid, a foam, a paste, a tablet, a gum, a solution, a baby formula, a baby toothpaste, a beverage, and a dairy product.

28. The method of claim 22, wherein the fatty acids/esters comprise about 0.01% to about 20% weight/weight of the composition.
